(19) United States
(12) Patent Application Publication
(10) Pub. No.: US 2005/0244478 A1
(43) Pub. Date: Nov. 3, 2005

(54) Anti-excititoxic sustained release intraocular implants and related methods

(75) Inventors: Patrick M. Hughes, Aliso Viejo, CA (US); James A. Burke, Santa Ana, CA (US); Rhett M. Schiffman, Laguna Beach, CA (US)

Correspondence Address: STOUT, UXA, BUYAN & MULLINS LLP, 4 VENTURE, SUITE 300, IRVINE, CA, 92618, US

(73) Assignee: Allergan, Inc., Irvine, CA (US)

(21) Appl. No.: 10/837,142

(22) Filed: Apr. 30, 2004

(51) Int. Cl. A61F 2/02

(52) U.S. Cl. 424/428

(57) ABSTRACT

Biocompatible intraocular implants include an anti-excitotoxic agent and a biodegradable polymer that is effective to facilitate release of the anti-excitotoxic agent into an eye for an extended period of time. The therapeutic agents of the implants may be associated with a biodegradable polymer matrix, such as a matrix that is substantially free of a polyvinyl alcohol. The implants may be placed in an eye to treat or reduce the occurrence of one or more ocular conditions, such as retinal damage, including glaucoma and proliferative vitreoretinopathy.

### BACKGROUND

The present invention generally relates to devices and methods to treat an eye of a patient, and more specifically to intraocular implants that provide extended release of a therapeutic agent to an eye in which the implant is placed, and to methods of making and using such implants, for example, to treat or reduce one or more symptoms of glaucoma, such as proliferative vitreoretinopathy and cellular damage or death.

Glaucoma affects approximately five percent of persons who are older than 65 years and fourteen percent of those older than 80 years. The visual loss which results from glaucoma conditions has been attributed to progressive damage of the optic nerve and consequent loss of retinal ganglion cells, mediated by elevated intraocular pressure (Quigley et al., Invest. Ophthalmol. Vis. Sci. 19:505, 1980). Consequently, therapeutic modalities have focused on the management of intraocular pressure.

Many compounds have been proposed to treat glaucoma. See generally, Horlington U.S. Pat. No. 4,425,346; Komuro et al. U.S. Pat. No. 4,396,625; Gubin et al. U.S. Pat. No. 5,017,579; Yamamori et al. U.S. Pat. No. 4,396,625; and Bodor et al. U.S. Pat. No. 4,158,005.

At the present time, medical control of intraocular pressure consists of topical or oral administration of a miotic (e.g., pilocarpine), epinephrine derivatives (e.g., dipivalyl epinephrine), carbonic anhydrase inhibitors, prostaglandins, prostamides, alpha adrenergic agents, or topical beta blockers (e.g., timolol). Abelson U.S. Pat. No. 4,981,871 discloses the use of a class I voltage-dependent Ca⁺⁺ channel blocking agent (a phenylalkylamine) to treat elevated ocular pressure (Specifically, Abelson '871 discloses the use of verapamil, which does not cross the blood brain barrier and does not reach retinal ganglion cells).

Miotics may reduce the patient's visual acuity, particularly in the presence of lenticular opacities. Topical beta blockers such as Timolol® have been associated with systemic side effects such as fatigue, confusion, or asthma, and exacerbation of cardiac symptoms has been reported after rapid withdrawal of topical beta blockers. Oral administration of carbonic anhydrase inhibitors, such as acetazolamide, may also be used, but these agents can be associated with systemic side effects including chronic metabolic acidosis.

If current methods of treatment fail to reduce intraocular pressure, laser treatment or a drainage operation (e.g., trabeculectomy) may be performed.

U.S. Pat. Nos. 5,922,773 and 6,482,854 disclose administration of a compound capable of reducing glutamate induced excitotoxicity in a concentration effective to cause reduction of such excitotoxicity.

U.S. Pat. No. 6,573,280 discloses administration of a compound to a patient to reduce glutamate-induced retinal cell migration to help treat proliferative vitreoretinopathy.

Neuroprotective effects of memantine are also described in a number of articles, see Woldemussie, “Neuroprotection of retinal ganglion cells in experimental models of glaucoma”, Minerva Oftalmol, 42(2):71-8 (2000); Wheeler, “Experimental studies of agents with potential neuroprotective properties”, Acta Ophthalmol Scand, 77(229):27-28 (1999); Schuettauf et al., “Effects of anti-glaucoma medications on ganglion cell survival: the DBA/2J mouse model”, Vision Res, 42(20):2333-7 (2002); WoldeMussie et al., “Neuroprotective effects of memantine in different retinal injury models in rats”, J Glaucoma 11 (6):474-480 (2002); and Hare et al., “Efficacy and safety of memantine, an NMDA-Type Open-Channel Blocker, for reduction of retinal injury associated with experimental glaucoma in rat and monkey”, Surv Ophthalmol 45(Suppl 3): S284-S289 (2001).

U.S. Pat. No. 6,713,081 discloses ocular implant devices made from polyvinyl alcohol and used for the delivery of a therapeutic agent to an eye in a controlled and sustained manner. The implants may be placed subconjunctivally or intravitreally in an eye.

Biocompatible implants for placement in the eye have also been disclosed in a number of patents, such as U.S. Pat. Nos. 4,521,210; 4,853,224; 4,997,652; 5,164,188; 5,443,505; 5,501,856; 5,766,242; 5,824,072; 5,869,079; 6,074,661; 6,331,313; 6,369,116; and 6,699,493.

It would be advantageous to provide eye implantable drug delivery systems, such as intraocular implants, and methods of using such systems, that are capable of releasing a therapeutic agent at a sustained or controlled rate for extended periods of time and in amounts with few or no negative side effects.

### SUMMARY

The present invention provides new drug delivery systems, and methods of making and using such systems, for extended or sustained drug release into an eye, for example, to achieve one or more desired therapeutic effects. The drug delivery systems are in the form of implants or implant elements that may be placed in an eye. The present systems and methods advantageously provide for extended release times of one or more therapeutic agents. Thus, the patient in whose eye the implant has been placed receives a therapeutic amount of an agent for a long or extended time period without requiring additional administrations of the agent. For example, the patient has a substantially consistent level of therapeutically active agent available for consistent treatment of the eye over a relatively long period of time, for example, on the order of at least about one week, such as between about two and about six months after receiving an implant. Such extended release times facilitate obtaining successful treatment results.

Intraocular implants in accordance with the disclosure herein comprise a therapeutic component and a drug release sustaining component associated with the therapeutic component. In accordance with the present invention, the therapeutic component comprises, consists essentially of, or consists of, a neuroprotective agent or an anti-excitoxicity agent. For example, the therapeutic component may comprise, consist essentially of, or consist of, one or more glutamate receptor antagonists, such as N-Methyl-D-Aspartate (NMDA) receptor antagonists, calcium channel blockers, and the like. The drug release sustaining component is associated with the therapeutic component to sustain release of an amount of the neuroprotective or anti-excitotoxic agent into an eye in which the implant is placed. The amount of the neuroprotective or anti-excitotoxic agent is released into the eye for a period of time greater than about one week after the implant is placed in the eye and is effective in reducing or treating an ocular condition, such as glaucoma, or other ocular conditions adversely affected by excitotoxicity.

In one embodiment, the intraocular implants comprise an NMDA receptor antagonist and a biodegradable polymer matrix that is substantially free of polyvinyl alcohol. The NMDA receptor antagonist is associated with a biodegradable polymer matrix that degrades at a rate effective to sustain release of an amount of the NMDA receptor antagonist from the implant effective to treat an ocular condition. The intraocular implant is biodegradable or bioerodible and provides a sustained release of the NMDA receptor antagonist in an eye for extended periods of time, such as for more than one week, for example for about three months or more and up to about six months or more. In certain implants, the NMDA receptor antagonist is memantine, salts thereof, and mixtures thereof.

The biodegradable polymer matrix of the foregoing implants may be a mixture of biodegradable polymers or the matrix may comprise a single type of biodegradable polymer. For example, the matrix may comprise a polymer selected from the group consisting of polylactides, poly(lactide-co-glycolides), and combinations thereof.

A method of making the present implants involves combining or mixing the anti-excitotoxic agent, such as the NMDA receptor antagonist, with a biodegradable polymer or polymers. The mixture may then be extruded or compressed to form a single composition. The single composition may then be processed to form individual implants suitable for placement in an eye of a patient.

The implants may be placed in an ocular region to treat a variety of ocular conditions, such as treating, preventing, or reducing at least one symptom associated with glaucoma, or ocular conditions related to excessive excitatory activity or glutamate receptor activation.

Kits in accordance with the present invention may comprise one or more of the present implants, and instructions for using the implants. For example, the instructions may explain how to administer the implants to a patient, and types of conditions that may be treated with the implants.

Each and every feature described herein, and each and every combination of two or more of such features, is included within the scope of the present invention provided that the features included in such a combination are not mutually inconsistent. In addition, any feature or combination of features may be specifically excluded from any embodiment of the present invention.

Additional aspects and advantages of the present invention are set forth in the following description and claims, particularly when considered in conjunction with the accompanying drawings.

### DESCRIPTION

As described herein, controlled and sustained administration of a therapeutic agent through the use of one or more intraocular implants may improve treatment of undesirable ocular conditions. The implants comprise a pharmaceutically acceptable polymeric composition and are formulated to release one or more pharmaceutically active agents, such as anti-excitotoxic agents or neuroprotective agents, including NMDA receptor antagonists, over an extended period of time. The implants are effective to provide a therapeutically effective dosage of the agent or agents directly to a region of the eye to treat, prevent, and/or reduce one or more symptoms of one or more undesirable ocular conditions. Thus, with a single administration, therapeutic agents will be made available at the site where they are needed and will be maintained for an extended period of time, rather than subjecting the patient to repeated injections or, in the case of self-administered drops, ineffective treatment with only limited bursts of exposure to the active agent or agents or, in the case of systemic administration, higher systemic exposure and concomitant side effects or, in the case of non-sustained release dosages, potentially toxic transient high tissue concentrations associated with pulsed, non-sustained release dosing.

An intraocular implant in accordance with the disclosure herein comprises a therapeutic component and a drug release sustaining component associated with the therapeutic component. In accordance with the present invention, the therapeutic component comprises, consists essentially of, or consists of, an anti-excitotoxic agent or neuroprotective agent, such as an NMDA receptor antagonist. The drug release sustaining component is associated with the therapeutic component to sustain release of an effective amount of the therapeutic component into an eye in which the implant is placed. The amount of the therapeutic component is released into the eye for a period of time greater than about one week after the implant is placed in the eye, and is effective in treating and/or reducing at least one symptom of one or more ocular conditions, such as neovascularization, angiogenesis, tumor growth, and the like.

Definitions

For the purposes of this description, we use the following terms as defined in this section, unless the context of the word indicates a different meaning.

As used herein, an “intraocular implant” refers to a device or element that is structured, sized, or otherwise configured to be placed in an eye. Intraocular implants are generally biocompatible with physiological conditions of an eye and do not cause unacceptable adverse side effects. Intraocular implants may be placed in an eye without disrupting vision of the eye.

As used herein, a “therapeutic component” refers to a portion of an intraocular implant comprising one or more therapeutic agents or substances used to treat a medical condition of the eye. The therapeutic component may be a discrete region of an intraocular implant, or it may be homogenously distributed throughout the implant. The therapeutic agents of the therapeutic component are typically ophthalmically acceptable, and are provided in a form that does not cause adverse reactions when the implant is placed in an eye.

As used herein, a “drug release sustaining component” refers to a portion of the intraocular implant that is effective to provide a sustained release of the therapeutic agents of the implant. A drug release sustaining component may be a biodegradable polymer matrix, or it may be a coating covering a core region of the implant that comprises a therapeutic component.

As used herein, “associated with” means mixed with, dispersed within, coupled to, covering, or surrounding.

As used herein, an “ocular region” or “ocular site” refers generally to any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, an “ocular condition” is a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

An anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, retinal pigmented epithelium, Bruch's membrane, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

The term “biodegradable polymer” refers to a polymer or polymers which degrade in vivo, and wherein erosion of the polymer or polymers over time occurs concurrent with or subsequent to release of the therapeutic agent. Specifically, hydrogels such as methylcellulose which act to release drug through polymer swelling are specifically excluded from the term “biodegradable polymer”. The terms “biodegradable” and “bioerodible” are equivalent and are used interchangeably herein. A biodegradable polymer may be a homopolymer, a copolymer, or a polymer comprising more than two different polymeric units.

The term “treat”, “treating”, or “treatment” as used herein, refers to reduction or resolution or prevention of an ocular condition, ocular injury or damage, or to promote healing of injured or damaged ocular tissue.

The term “therapeutically effective amount” as used herein, refers to the level or amount of agent needed to treat an ocular condition, or reduce or prevent ocular injury or damage without causing significant negative or adverse side effects to the eye or a region of the eye.

Intraocular implants have been developed which can release drug loads over various' time periods. These implants, which when inserted into an eye, such as the vitreous of an eye, provide therapeutic levels of an anti-excitotoxic agent or neuroprotective agent, such as an NMDA receptor antagonist, for extended periods of time (e.g., for about 1 week or more). The disclosed implants are effective in treating ocular conditions, such as posterior ocular conditions, such as glaucoma.

In one embodiment of the present invention, an intraocular implant comprises a biodegradable polymer matrix. The biodegradable polymer matrix is one type of a drug release sustaining component. The biodegradable polymer matrix is effective in forming a biodegradable intraocular implant. The biodegradable intraocular implant comprises an NMDA receptor antagonist associated with the biodegradable polymer matrix. The matrix degrades at a rate effective to sustain release of an amount of the NMDA receptor antagonist for a time greater than about one week from the time in which the implant is placed in ocular region or ocular site, such as the vitreous of an eye.

The NMDA receptor antagonist of the implant is typically an agent that reduces neuronal damage mediated by the NMDA receptor complex. Examples of NMDA receptor antagonist useful in the present implants are described in U.S. Pat. Nos. 5,922,773, 6,482,854; and 6,573,280. In short, an NMDA receptor antagonist of the present implants refers to channel blockers (e.g., antagonists that operate uncompetitively to block the NMDA receptor channel); receptor antagonists (e.g., antagonists that compete with NMDA or glutamate to act at the NMDA or glutamate binding site); agents acting at either the glycine co-agonist site or any of several modulation sites, such as the zinc site, the magnesium site, the redox modulatory site, or the polyamine site; or agents that inhibit the downstream effects of NMDA receptor stimulation, such as agents that inhibit activation of protein kinase C activation by NMDA or glutamate stimulation, antioxidants, and agents that decrease phosphatidyl metabolism. Some specific examples of antiexcitotoxic agents include amantadine derivates, salts thereof, and combinations thereof. For example, the amantadine derivates may be memantine, amantadine, and rimantadine. Other antiexcitotoxic agents may include nitroglycerin, dextorphan, dextromethorphan, and CGS-19755. Some compounds include those in the following table

| NMDA Antagonists | NMDA Antagonists | NMDA Antagonists |
|---|---|---|
| 1. Competitive NMDA | 2. Channel Blockers (Un- | 3. Antagonists at Glydne Site of |
| Antagonists (act at agonist | Competitve NMDA Antagonists) | the NMDA Receptor |
| binding site) | MK-801 (Dizocilpine) and other | Kynurenate, 7-chloro- |
| CGS-19755 (CIBA-GEIGY) and | derivatives of | kynurenate, 5,7-chloro- |
| other piperdine derivatives, D-2- | dibenzyocycloheptene (Merck) | kynurenate, thio-derivatives, |
| amino-5-phosphovalerate, D-2- | Sigma receptor ligands, e.g. | and other derivatives. (Merck) |
| amino-7-phosphosoheptanoate | Dextrorphan, dextromethorphan | Indole-2-carboxylic acid |
| (AP7) CPP {[3-2- | and morphiasn derivatives | DNQX |
| carboxypiperazin-4-y-propyl-1- | (Hoffman La Roche) such as | Quinoxaline or oxidiazole |
| phosphonic acid]} | caramiphen and rimcazole | derivatives including CNQX, |
| LY 274614, CGP39551, | (which also block calcium | NBQX |
| CGP37849, LY233053, | channels) | Glycine partial agonist (e.g. |
| LY233536 | Ketamine, Tiletamine and other | Hoecht-Roussel P-9939 |
| O-phosphohomoserine | cyclohexanes | 6. Other Non-Competitve |
| MDL100,453 | Phencyclidine (PCP) and | NMDA Antagonists |
| 4. Polyamine Site of NMDA | derivatives, and pyrazine | Hoechst 831917189 |
| Receptor | compounds | SKB Carvedilol |
| Arcaine and relate biguanidines | Memantine, amantadine, | |
| and | rimantadine and derivatives | |
| biogenic polyamines | CNS 1102 (and related bi- and | |
| Ifenprodil and related drugs | tri-substituted guanidines) | |
| Diethylenetriamine SL 82,0715 | Diamines | |
| 1,10-diaminodecane (and | Conantokan peptide from | |
| related inverse agonists) | Conus geographus | |
| | Agatoxis-489 | |
| | 5. Redox Site of NMDA | |
| | Receptor | |
| | Oxidized and reduced | |
| | glutathione | |
| | PQQ (pyrroloquinoline quinone) | |
| | Compounds that generate Nitric | |
| | Oxide (NO) or other oxidation | |
| | states of nitrogen monoxide | |
| | (NO+, NO−) including those | |
| | listed in the box below | |
| | Nitroglycerin and derivatives, | |
| | Sodium Nitroprusside, and | |
| | other NO generating listed on | |
| | p.5 of this table | |
| | Nitric oxide synthase (NOS) | |
| | Inhibitors: | |
| | Arginise analogs including N- | |
| | mono-methyl-L-arginine (NMA); | |
| | N-amino-L-arginine (NAA); N- | |
| | nitro-L arginine (NNA); N-nitro- | |
| | L-arginine methyl ester; N- | |
| | iminoethyl-L-omithine | |
| | Flavin inhibitors; | |
| | diphenyliodinium; Calmoduli | |
| | inhibitors, trifluoperizine | |
| | Calcineurin Inhibitors, e.g., FK- | |
| | 506 (inhibits calcineurin and | |
| | thus NOS diphosphorylase) | |
| Inhibitors of Downstream | Inhibitors of Downstream | Non-NMDA Receptor |
| Effects of NMDA | Effects of NMDA | Antagonists |
| 7. Agents to inhibit protein | 8. Downstream effects from | 9A. Non-NMDA antagonists |
| kinase C activation by NMDA | Receptor Activation | (Competitive) |
| stimulation (Involved in NMDA | 8a. To decrease | CNQX, NBQX, YM900, DNQX. |
| toxicity) | phosphatidylinositol metabolism | PD140532 |
| MDL 27,266 (Merrill Dow) and | kappa opioid receptor agonist: | AMOA (2-amino-3[3- |
| triazoleone derivatives | U50488 (Upjohn) and | 9carboxymethoxyl-5- |
| Mososialoganglioxides (eg GMI | dynorphan | methoxylisoxazol-4- |
| of Fidin Corp.) and other | kapp opioid receptor agonist: | yl]propionate] |
| ganglioside derivatives LIGA20, | PD117302, CI-977 | 2-phosphophonoethyl |
| LIGA4 (may also affect calcium | 8b. To decrease hydrogen | phenylalanine derivatives, i.e., |
| extrusion via calcium ATPase) | peroxide and free radical injury, | 5-ethyl, 5-methyl, 5- |
| | eg antioxidants 21-aminosteroid | trifluoromethyl |
| | (lazaroids) such as U74500A, | 9B. Non-NMDA Non |
| | U75412E and U74006F | competitive antagonists |
| | U74389F, FLE26749, Trolox | GYK152466 |
| | (water soluble alpha | Evans Blue |
| | tocophenol), 3,5-dialkoxy-4- | |
| | hydroxy-benzylamines | |
| | Compounds that generate Nitric | |
| | Oxide (NO) or other oxidation | |
| | states of nitrogen monoxide | |
| | (NO+, NO−) including those | |
| | listed in the box below | |
| | Nitroglycerin and derivatives, | |
| | Sodium Nitroprusside, and | |
| | other NO generating listied on | |
| | p.5 of this table | |
| | Nitric oxide Synthase (NOS) | |
| | Inhibition: | |
| | Arginine analogs including N- | |
| | mono-methyl-L-arginine (NMA); | |
| | N-amino-L-arginine (NAA); N- | |
| | nitro-L arginine (NNA); N-nitro- | |
| | L-arginine methyl ester; N- | |
| | iminoethyl-L-omithine | |
| | | Drugs to decrease intracellular |
| Agents Active at Metabotropic | | calcium following glutamate |
| Glutamate Receptors | Decrease Glutamate Release | receptor stimulation |
| 10a. Blockers of Metabotropic | 11. Agents to decrease | 12a. Agents to decrease |
| Glutamate Receptors | glutamate release | Intracellular calcium release |
| AP3 (2-amino-3- | Adenosine, and derivatives, | Dantrolen (sodium dantrium: |
| phosphonoprionic acid) | e.g., cyclohexyladenosine | Ryanodine (or |
| 10b. Agonists of Metabotropic | CN51145 | ryanodine + caffeine) |
| Glutamate Receptors (1S, 3R)- | Conopeptides: SNX-111, SNX- | 12b. Agents Inhibiting |
| 1-Amino-cyclopentane-1,3- | 183, SNX-230 | intracellular Calcium-ATPase |
| dicarboxylic acid [(1S, 3R)- | Omega-Aga-IVA, toxin from | Thaprigargin, cyclopiazosic |
| ACPD], commonly referred to | venom of funnel spider | acid, BHQ ([2,5-di-(tert butyl)- |
| as ‘trans’-ACPD | Compounds that generate Nitric | 1,4-benzohydroquinose]) |
| | Oxide (NO) or other oxidation | |
| | states of nitrogen monoxide | |
| | (NO+, NO−)including those | |
| | listed in the box below | |
| | Nitroglycerin and derivatives, | |
| | Sodium Nitroprusside, and | |
| | other NO generating listied on | |
| | p.5 of this table | |
| | Nitric oxide Synthase (NOS) | |
| | Inhibitors: | |
| | Arginine analogs including N- | |
| | mono-methyl-L-arginine (NMA); | |
| | N-amino-L-arginine (NAA); N- | |
| | nitro-L arginine (NNA); N-nitro- | |
| | L-arginine methyl ester; N- | |
| | iminoethyl-L-omithine | |
| | Additional NO-generating | |
| | compounds | |
| | Isosorbide dinitrate (isordil) | |
| | S-nitrosocaptopril (SnoCap) | |
| | Serum albumin coupled to nitric | |
| | oxide (SA-NO) | |
| | Cathepsin coupled to nitric | |
| | oxide (cathepsin-NO) | |
| | Tissue plasminogen activator | |
| | coupled to NO (TPA-NO) | |
| | SIN-1 (also known as SIN1 or | |
| | molsidonmine) | |
| | Ion-nitrosyl complexes (e.g., | |
| | nitrosyl-iron complexes, with | |
| | iron in the Fe²⁺ state) | |
| | Nicorandil | |

These implants may also include salts of the NMDA receptor antagonists. Pharmaceutically acceptable acid addition salts of the compounds of the invention are those formed from acids which form non-toxic addition salts containing pharmaceutically acceptable anions, such as the hydrochloride, hydrobromide, hydroiodide, sulfate, or bisulfate, phosphate or acid phosphate, acetate, maleate, fumarate, oxalate, lactate, tartrate, citrate, gluconate, saccharate and p-toluene sulphonate salts.

Thus, the implant may comprise a therapeutic component which comprises, consists essentially of, or consists of an NMDA receptor antagonist, such as memantine, salts thereof, and mixtures thereof. The biodegradable polymer matrix of such implants is preferably substantially free of polyvinyl alcohol, or in other words, includes no polyvinyl alcohol.

Additional antiexcitotoxic agents may be obtained using conventional methods, such as by routine chemical synthesis methods known to persons of ordinary skill in the art. Therapeutically effective antiexcitotoxic agents may be screened and identified using conventional screening technologies, for example, by determining the amount of cell death in a conventional toxicity assay, or by other assays which may be used in identifying the effectiveness of the compounds above.

The antiexcitotoxic agents, such as the NMDA receptor antagonists, may be in a particulate or powder form and entrapped by the biodegradable polymer matrix. Usually, antiexcitotoxic agent particles in intraocular implants will have an effective average size less than about 3000 nanometers. In certain implants, the particles may have an effective average particle size about an order of magnitude smaller than 3000 nanometers. For example, the particles may have an effective average particle size of less than about 500 nanometers. In additional implants, the particles may have an effective average particle size of less than about 400 nanometers, and in still further embodiments, a size less than about 200 nanometers.

The antiexcitotoxic agent of the implant is preferably from about 10% to 90% by weight of the implant. More preferably, the antiexcitotoxic agent is from about 20% to about 80% by weight of the implant. In a preferred embodiment, the antiexcitotoxic agent comprises about 40% by weight of the implant (e.g., 30%-50%). In another embodiment, the antiexcitotoxic agent comprises about 60% by weight of the implant.

Suitable polymeric materials or compositions for use in the implant include those materials which are compatible, that is biocompatible, with the eye so as to cause no substantial interference with the functioning or physiology of the eye. Such materials preferably are at least partially and more preferably substantially completely biodegradable or bioerodible.

Examples of useful polymeric materials include, without limitation, such materials derived from and/or including organic esters and organic ethers, which when degraded result in physiologically acceptable degradation products, including the monomers. Also, polymeric materials derived from and/or including, anhydrides, amides, orthoesters and the like, by themselves or in combination with other monomers, may also find use. The polymeric materials may be addition or condensation polymers, advantageously condensation polymers. The polymeric materials may be cross-linked or non-cross-linked, for example not more than lightly cross-linked, such as less than about 5%, or less than about 1% of the polymeric material being cross-linked. For the most part, besides carbon and hydrogen, the polymers will include at least one of oxygen and nitrogen, advantageously oxygen. The oxygen may be present as oxy, e.g. hydroxy or ether, carbonyl, e.g. non-oxo-carbonyl, such as carboxylic acid ester, and the like. The nitrogen may be present as amide, cyano and amino. The polymers set forth in Heller, Biodegradable Polymers in Controlled Drug Delivery, In: CRC Critical Reviews in Therapeutic Drug Carrier Systems, Vol. 1, CRC Press, Boca Raton, Fla. 1987, pp 39-90, which describes encapsulation for controlled drug delivery, may find use in the present implants.

Of additional interest are polymers of hydroxyaliphatic carboxylic acids, either homopolymers or copolymers, and polysaccharides. Polyesters of interest include polymers of D-lactic acid, L-lactic acid, racemic lactic acid, glycolic acid, polycaprolactone, and combinations thereof. Generally, by employing the L-lactate or D-lactate, a slowly eroding polymer or polymeric material is achieved, while erosion is substantially enhanced with the lactate racemate.

Among the useful polysaccharides are, without limitation, calcium alginate, and functionalized celluloses, particularly carboxymethylcellulose esters characterized by being water insoluble, a molecular weight of about 5 kD to 500 kD, for example.

Other polymers of interest include, without limitation, polyesters, polyethers and combinations thereof which are biocompatible and may be biodegradable and/or bioerodible.

Some preferred characteristics of the polymers or polymeric materials for use in the present invention may include biocompatibility, compatibility with the therapeutic component, ease of use of the polymer in making the drug delivery systems of the present invention, a half-life in the physiological environment of at least about 6 hours, preferably greater than about one day, not significantly increasing the viscosity of the vitreous, and water insolubility.

The biodegradable polymeric materials which are included to form the matrix are desirably subject to enzymatic or hydrolytic instability. Water soluble polymers may be cross-linked with hydrolytic or biodegradable unstable cross-links to provide useful water insoluble polymers. The degree of stability can be varied widely, depending upon the choice of monomer, whether a homopolymer or copolymer is employed, employing mixtures of polymers, and whether the polymer includes terminal acid groups.

Equally important to controlling the biodegradation of the polymer and hence the extended release profile of the implant is the relative average molecular weight of the polymeric composition employed in the implant. Different molecular weights of the same or different polymeric compositions may be included in the implant to modulate the release profile. In certain implants, the relative average molecular weight of the polymer will range from about 9 to about 64 kD, usually from about 10 to about 54 kD, and more usually from about 12 to about 45 kD.

In some implants, copolymers of glycolic acid and lactic acid are used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the implant, where a more flexible implant is desirable for larger geometries. The % of polylactic acid in the polylactic acid polyglycolic acid (PLGA) copolymer can be 0-100%, preferably about 15-85%, more preferably about 35-65%. In some implants, a 50/50 PLGA copolymer is used.

The biodegradable polymer matrix of the intraocular implant may comprise a mixture of two or more biodegradable polymers. For example, the implant may comprise a mixture of a first biodegradable polymer and a different second biodegradable polymer. One or more of the biodegradable polymers may have terminal acid groups.

Release of a drug from an erodible polymer is the consequence of several mechanisms or combinations of mechanisms. Some of these mechanisms include desorption from the implants surface, dissolution, diffusion through porous channels of the hydrated polymer and erosion. Erosion can be bulk or surface or a combination of both. As discussed herein, the matrix of the intraocular implant may release drug at a rate effective to sustain release of an amount of the antiexcitotoxic agents for more than one week after implantation into an eye. In certain implants, therapeutic amounts of the antiexcitotoxic agents are released for more than about one month, and even for about six months or more.

One example of the biodegradable intraocular implant comprises memantine associated with a biodegradable polymer matrix that is substantially free of polyvinyl alcohol, and comprises a poly(lactide-co-glycolide) or a poly(D,L-lactide-co-glycolide). The implant may have an amount of memantine from about 40% to about 70% by weight of the implant. Such a mixture is effective in sustaining release of a therapeutically effective amount of the memantine for a time period from about two months to about four months from the time the implant is placed in an eye.

The release of the antiexcitotoxic agent(s) from the intraocular implant comprising a biodegradable polymer matrix may include an initial burst of release followed by a gradual increase in the amount of the antiexcitotoxic agent(s) released, or the release may include an initial delay in release of the antiexcitotoxic agent(s) followed by an increase in release. When the implant is substantially completely degraded, the percent of the antiexcitotoxic agent(s) that has been released is about one hundred. Compared to existing implants, the implants disclosed herein do not completely release, or release about 100% of the antiexcitotoxic agent(s), until after about one week of being placed in an eye.

It may be desirable to provide a relatively constant rate of release of the antiexcitotoxic agent(s) from the implant over the life of the implant. For example, it may be desirable for the antiexcitotoxic agent(s) to be released in amounts from about 0.01 μg to about 2 μg per day for the life of the implant. However, the release rate may change to either increase or decrease depending on the formulation of the biodegradable polymer matrix. In addition, the release profile of the antiexcitotoxic agent(s) may include one or more linear portions and/or one or more non-linear portions. Preferably, the release rate is greater than zero once the implant has begun to degrade or erode.

The implants may be monolithic, i.e. having the active agent or agents homogenously distributed through the polymeric matrix, or encapsulated, where a reservoir of active agent is encapsulated by the polymeric matrix. Due to ease of manufacture, monolithic implants are usually preferred over encapsulated forms. However, the greater control afforded by the encapsulated, reservoir-type implant may be of benefit in some circumstances, where the therapeutic level of the drug falls within a narrow window. In addition, the therapeutic component, including the antiexcitotoxic agent(s), may be distributed in a non-homogenous pattern in the matrix. For example, the implant may include a portion that has a greater concentration of the antiexcitotoxic agent(s) relative to a second portion of the implant.

The intraocular implants disclosed herein may have a size of between about 5 μm and about 2 mm, or between about 10 μm and about 1 mm for administration with a needle, greater than 1 mm, or greater than 2 mm, such as 3 mm or up to 10 mm, for administration by surgical implantation. The vitreous chamber in humans is able to accommodate relatively large implants of varying geometries, having lengths of, for example, 1 to 10 mm. The implant may be a cylindrical pellet (e.g., rod) with dimensions of about 2 mm×0.75 mm diameter. Or the implant may be a cylindrical pellet with a length of about 7 mm to about 10 mm, and a diameter of about 0.75 mm to about 1.5 mm.

The implants may also be at least somewhat flexible so as to facilitate both insertion of the implant in the eye, such as in the vitreous, and accommodation of the implant. The total weight of the implant is usually about 250-5000 μg, more preferably about 500-1000 μg. For example, an implant may be about 500 μg, or about 1000 μg. For non-human individuals, the dimensions and total weight of the implant(s) may be larger or smaller, depending on the type of individual. For example, humans have a vitreous volume of approximately 3.8 ml, compared with approximately 30 ml for horses, and approximately 60-100 ml for elephants. An implant sized for use in a human may be scaled up or down accordingly for other animals, for example, about 8 times larger for an implant for a horse, or about, for example, 26 times larger for an implant for an elephant.

Thus, implants can be prepared where the center may be of one material and the surface may have one or more layers of the same or a different composition, where the layers may be cross-linked, or of a different molecular weight, different density or porosity, or the like. For example, where it is desirable to quickly release an initial bolus of drug, the center may be a polylactate coated with a polylactate-polyglycolate copolymer, so as to enhance the rate of initial degradation. Alternatively, the center may be polyvinyl alcohol coated with polylactate, so that upon degradation of the polylactate exterior the center would dissolve and be rapidly washed out of the eye.

The implants may be of any geometry including fibers, sheets, films, microspheres, spheres, circular discs, plaques and the like. The upper limit for the implant size will be determined by factors such as toleration for the implant, size limitations on insertion, ease of handling, etc. Where sheets or films are employed, the sheets or films will be in the range of at least about 0.5 mm×0.5 mm, usually about 3-10 mm×5-10 mm with a thickness of about 0.1-1.0 mm for ease of handling. Where fibers are employed, the fiber diameter will generally be in the range of about 0.05 to 3 mm and the fiber length will generally be in the range of about 0.5-10 mm. Spheres may be in the range of about 0.5 μm to 4 mm in diameter, with comparable volumes for other shaped particles.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant are chosen to suit the site of implantation.

The proportions of antiexcitotoxic agent(s), polymer, and any other modifiers may be empirically determined by formulating several implants with varying proportions. A USP approved method for dissolution or release test can be used to measure the rate of release (USP 23; NF 18 (1995) pp. 1790-1798). For example, using the infinite sink method, a weighed sample of the implant is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to the antiexcitotoxic agent(s) included in the intraocular implants disclosed herein, the intraocular implants may also include one or more additional ophthalmically acceptable therapeutic agents. For example, the implant may include one or more antihistamines, one or more antibiotics, one or more beta blockers, one or more steroids, one or more antineoplastic agents, one or more immunosuppressive agents, one or more antiviral agents, one or more antioxidant agents, and mixtures thereof.

Pharmacologic or therapeutic agents which may find use in the present systems, include, without limitation, those disclosed in U.S. Pat. No. 4,474,451, columns 4-6 and U.S. Pat. No. 4,327,725, columns 7-8.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin,, cefamandole, cefoxitin, cefonicid, ceforanide, ceftriaxone, cefadroxil, cephradine, cefuroxime, cyclosporine, ampicillin, amoxicillin, cyclacillin, ampicillin, penicillin G, penicillin V potassium, piperacillin, oxacillin, bacampicillin, cloxacillin, ticarcillin, azlocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, gatifloxacin, ofloxacin, and derivatives thereof.

Examples of beta blockers include acebutolol, atenolol, labetalol, metoprolol, propranolol, timolol, and derivatives thereof.

Examples of steroids include corticosteroids, such as cortisone, prednisolone, flurometholone, dexamethasone, medrysone, loteprednol, fluazacort, hydrocortisone, prednisone, betamethasone, prednisone, methylprednisolone, riamcinolone hexacatonide, paramethasone acetate, diflorasone, fluocinonide, fluocinolone, triamcinolone, derivatives thereof, and mixtures thereof.

Examples of antineoplastic agents include adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppresive agents include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir and derivatives thereof.

Examples of antioxidant agents include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Other therapeutic agents include squalamine, carbonic anhydrase inhibitors, alpha agonists, prostamides, prostaglandins, antiparasitics, antifungals, and derivatives thereof.

The amount of active agent or agents employed in the implant, individually or in combination, will vary widely depending on the effective dosage required and the desired rate of release from the implant. As indicated herein, the agent will be at least about 1, more usually at least about 10 weight percent of the implant, and usually not more than about 80, more usually not more than about 40 weight percent of the implant.

In addition to the therapeutic component, the intraocular implants disclosed herein may include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from 0.001 to about 5% by weight and preferably 0.01 to about 2% by weight.

In addition, the implants may include a solubility enhancing component provided in an amount effective to enhance the solubility of the antiexcitotoxic agent(s) relative to substantially identical implants without the solubility enhancing component. For example, an implant may include a β-cyclodextrin, which is effective in enhancing the solubility of the anti-excitotoxic agent. The β-cyclodextrin may be provided in an amount from about 0.5% (w/w) to about 25% (w/w) of the implant. In certain implants, the β-cyclodextrin is provided in an amount from about 5% (w/w) to about 15% (w/w) of the implant

In some situations mixtures of implants may be utilized employing the same or different pharmacological agents. In this way, a cocktail of release profiles, giving a biphasic or triphasic release with a single administration is achieved, where the pattern of release may be greatly varied.

Additionally, release modulators such as those described in U.S. Pat. No. 5,869,079 may be included in the implants. The amount of release modulator employed will be dependent on the desired release profile, the activity of the modulator, and on the release profile of the antiexcitotoxic agent(s) in the absence of modulator. Electrolytes such as sodium chloride and potassium chloride may also be included in the implant. Where the buffering agent or enhancer is hydrophilic, it may also act as a release accelerator. Hydrophilic additives act to increase the release rates through faster dissolution of the material surrounding the drug particles, which increases the surface area of the drug exposed, thereby increasing the rate of drug bioerosion. Similarly, a hydrophobic buffering agent or enhancer dissolve more slowly, slowing the exposure of drug particles, and thereby slowing the rate of drug bioerosion.

Various techniques may be employed to produce the implants described herein. Useful techniques include, but are not necessarily limited to, solvent evaporation methods, phase separation methods, interfacial methods, molding methods, injection molding methods, extrusion methods, co-extrusion methods, carver press method, die cutting methods, heat compression, combinations thereof and the like.

Specific methods are discussed in U.S. Pat. No. 4,997,652. Extrusion methods may be used to avoid the need for solvents in manufacturing. When using extrusion methods, the polymer and drug are chosen so as to be stable at the temperatures required for manufacturing, usually at least about 85 degrees Celsius. Extrusion methods use temperatures of about 25 degrees C. to about 150 degrees C., more preferably about 65 degrees C. to about 130 degrees C. An implant may be produced by bringing the temperature to about 60 degrees C. to about 150 degrees C. for drug/polymer mixing, such as about 130 degrees C., for a time period of about 0 to 1 hour, 0 to 30 minutes, or 5-15 minutes. For example, a time period may be about 10 minutes, preferably about 0 to 5 min. The implants are then extruded at a temperature of about 60 degrees C. to about 130 degrees C., such as about 75 degrees C.

In addition, the implant may be coextruded so that a coating is formed over a core region during the manufacture of the implant.

Compression methods may be used to make the implants, and typically yield implants with faster release rates than extrusion methods. Compression methods may use pressures of about 50-150 psi, more preferably about 70-80 psi, even more preferably about 76 psi, and use temperatures of about 0 degrees C. to about 115 degrees C., more preferably about 25 degrees C.

The implants of the present invention may be inserted into the eye, for example the vitreous chamber of the eye, by a variety of methods, including placement by forceps or by trocar following making a 2-3 mm incision in the sclera. One example of a device that may be used to insert the implants into an eye is disclosed in U.S. Patent Publication No. 2004/0054374. The method of placement may influence the therapeutic component or drug release kinetics. For example, delivering the implant with a trocar may result in placement of the implant deeper within the vitreous than placement by forceps, which may result in the implant being closer to the edge of the vitreous. The location of the implant may influence the concentration gradients of therapeutic component or drug surrounding the element, and thus influence the release rates (e.g., an element placed closer to the edge of the vitreous may result in a slower release rate).

The present implants are configured to release an amount of the antiexcitotoxic agent(s) effective to treat or reduce a symptom of an ocular condition, such as an ocular condition related to excessive glutamate activity or excitotoxicity, such as glaucoma. More specifically, the implants may be used in a method to tread or reduce one or more symptoms of glaucoma or proliferative vitreoretinopathy.

The implants disclosed herein may also be configured to release the antiexcitotoxic agent(s) or additional therapeutic agents, as described above, which to prevent diseases or conditions, such as the following:

MACULOPATHIES/RETINAL DEGENERATION: Non-Exudative Age Related Macular Degeneration (ARMD), Exudative Age Related Macular Degeneration (ARMD), Choroidal Neovascularization, Diabetic Retinopathy, Acute Macular Neuroretinopathy, Central Serous Chorioretinopathy, Cystoid Macular Edema, Diabetic Macular Edema.

UVEITIS/RETINITIS/CHOROIDITIS: Acute Multifocal Placoid Pigment Epitheliopathy, Behcet's Disease, Birdshot Retinochoroidopathy, Infectious (Syphilis, Lyme, Tuberculosis, Toxoplasmosis), Intermediate Uveitis (Pars Planitis), Multifocal Choroiditis, Multiple Evanescent White Dot Syndrome (MEWDS), Ocular Sarcoidosis, Posterior Scleritis, Serpignous Choroiditis, Subretinal Fibrosis and Uveitis Syndrome, Vogt-Koyanagi-Harada Syndrome.

VASCULAR DISEASES/EXUDATIVE DISEASES: Coat's Disease, Parafoveal Telangiectasis, Papillophlebitis, Frosted Branch Angitis, Sickle Cell Retinopathy and other Hemoglobinopathies, Angioid Streaks, Familial Exudative Vitreoretinopathy.

TRAUMATIC/SURGICAL: Sympathetic Ophthalmia, Uveitic Retinal Disease, Retinal Detachment, Trauma, Laser, PDT, Photocoagulation, Hypoperfusion During Surgery, Radiation Retinopathy, Bone Marrow Transplant Retinopathy.

PROLIFERATIVE DISORDERS: Proliferative Vitreal Retinopathy and Epiretinal Membranes, Proliferative Diabetic Retinopathy, Retinopathy of Prematurity (retrolental fibroplastic).

INFECTIOUS DISORDERS: Ocular Histoplasmosis, Ocular Toxocariasis, Presumed Ocular Histoplasmosis Syndrome (POHS), Endophthalmitis, Toxoplasmosis, Retinal Diseases Associated with HIV Infection, Choroidal Disease Associated with HIV Infection, Uveitic Disease Associated with HIV Infection, Viral Retinitis, Acute Retinal Necrosis, Progressive Outer Retinal Necrosis, Fungal Retinal Diseases, Ocular Syphilis, Ocular Tuberculosis, Diffuse Unilateral Subacute Neuroretinitis, Myiasis.

GENETIC DISORDERS: Systemic Disorders with Accosiated Retinal Dystrophies, Congenital Stationary Night Blindness, Cone Dystrophies, Fundus Flavimaculatus, Best's Disease, Pattern Dystrophy of the Retinal Pigmented Epithelium, X-Linked Retinoschisis, Sorsby's Fundus Dystrophy, Benign Concentric Maculopathy, Bietti's Crystalline Dystrophy, pseudoxanthoma elasticum, Osler Weber syndrome.

RETINAL TEARS/HOLES: Retinal Detachment, Macular Hole, Giant Retinal Tear.

TUMORS: Retinal Disease Associated with Tumors, Solid Tumors, Tumor Metastasis, Benign Tumors, for example, hemangiomas, neurofibromas, trachomas, and pyogenic granulomas, Congenital Hypertrophy of the RPE, Posterior Uveal Melanoma, Choroidal Hemangioma, Choroidal Osteoma, Choroidal Metastasis, Combined Hamartoma of the Retina and Retinal Pigmented Epithelium, Retinoblastoma, Vasoproliferative Tumors of the Ocular Fundus, Retinal Astrocytoma, Intraocular Lymphoid Tumors.

MISCELLANEOUS: Punctate Inner Choroidopathy, Acute Posterior Multifocal Placoid Pigment Epitheliopathy, Myopic Retinal Degeneration, Acute Retinal Pigment Epithelitis, Ocular inflammatory and immune disorders, ocular vascular malfunctions, Corneal Graft Rejection, Neovascular Glaucoma and the like.

In one embodiment, an implant, such as the implants disclosed herein, is administered to a posterior segment of an eye of a human or animal patient, and preferably, a living human or animal. In at least one embodiment, an implant is administered without accessing the subretinal space of the eye. For example, a method of treating a patient may include placing the implant directly into the posterior chamber of the eye. In other embodiments, a method of treating a patient may comprise administering an implant to the patient by at least one of intravitreal injection, subconjuctival injection, sub-tenon injections, retrobulbar injection, and suprachoroidal injection.

In at least one embodiment, a method of reducing neovascularization or angiogenesis in a patient comprises administering one or more implants containing one or more antiexcitotoxic agents, as disclosed herein to a patient by at least one of intravitreal injection, subconjuctival injection, sub-tenon injection, retrobulbar injection, and suprachoroidal injection. A syringe apparatus including an appropriately sized needle, for example, a 22 gauge needle, a 27 gauge needle or a 30 gauge needle, can be effectively used to inject the composition with the posterior segment of an eye of a human or animal. Repeat injections are often not necessary due to the extended release of the anti-excitotoxic agent from the implants.

In another aspect of the invention, kits for treating an ocular condition of the eye are provided, comprising: a) a container comprising an extended release implant comprising a therapeutic component including an antiexcitotoxic agent, such as an NMDA receptor antagonist (e.g., memantine), and a drug release sustaining component; and b) instructions for use. Instructions may include steps of how to handle the implants, how to insert the implants into an ocular region, and what to expect from using the implants.

### EXAMPLE 1

### Manufacture and Testing of Implants Containing an Nmda Receptor Antagonist and a Biodegradable Polymer Matrix

Biodegradable implants are made by combining memantine with a biodegradable polymer composition in a stainless steel mortar. The combination is mixed via a Turbula shaker set at 96 RPM for 15 minutes. The powder blend is scraped off the wall of the mortar and then remixed for an additional 15 minutes. The mixed powder blend is heated to a semi-molten state at specified temperature for a total of 30 minutes, forming a polymer/drug melt.

Rods are manufactured by pelletizing the polymer/drug melt using a 9 gauge polytetrafluoroethylene (PTFE) tubing, loading the pellet into the barrel and extruding the material at the specified core extrusion temperature into filaments. The filaments are then cut into about 1 mg size implants or drug delivery systems. The rods have dimensions of about 2 mm long×0.72 mm diameter. The rod implants weigh between about 900 μg and 1100 μg.

Wafers are formed by flattening the polymer melt with a Carver press at a specified temperature and cutting the flattened material into wafers, each weighing about 1 mg. The wafers have a diameter of about 2.5 mm and a thickness of about 0.13 mm. The wafer implants weigh between about 900 μg and 1100 μg.

In-vitro release testing can be performed on each lot of implant (rod or wafer). Each implant may be placed into a 24 mL screw cap vial with 10 mL of Phosphate Buffered Saline solution at 37° C. and 1 mL aliquots are removed and replaced with equal volume of fresh medium on day 1, 4, 7, 14, 28, and every two weeks thereafter.

Drug assays may be performed by HPLC, which consists of a Waters 2690 Separation Module (or 2696), and a Waters 2996 Photodiode Array Detector. An Ultrasphere, C-18 (2), 5 μm; 4.6×150 mm column heated at 30° C. can be used for separation and the detector can be set at 264 nm. The mobile phase can be (10:90) MeOH—buffered mobile phase with a flow rate of 1 mL/min and a total run time of 12 min per sample. The buffered mobile phase may comprise (68:0.75:0.25:31) 13 mM 1-Heptane Sulfonic Acid, sodium salt—glacial acetic acid—triethylamine—Methanol. The release rates can be determined by calculating the amount of drug being released in a given volume of medium over time in μg/day.

The polymers chosen for the implants can be obtained from Boehringer Ingelheim or Purac America, for example. Examples of polymers include: RG502, RG752, R202H, R203 and R206, and Purac PDLG (50/50). RG502 is (50:50) poly(D,L-lactide-co-glycolide), RG752 is (75:25) poly(D,L-lactide-co-glycolide), R202H is 100% poly(D, L-lactide) with acid end group or terminal acid groups, R203 and R206 are both 100% poly(D, L-lactide). Purac PDLG (50/50) is (50:50) poly(D,L-lactide-co-glycolide). The inherent viscosity of RG502, RG752, R202H, R203, R206, and Purac PDLG are 0.2, 0.2, 0.2, 0.3, 1.0, and 0.2 dL/g, respectively. The average molecular weight of RG502, RG752, R202H, R203, R206, and Purac PDLG are, 11700, 11200, 6500, 14000, 63300, and 9700 daltons, respectively.

### EXAMPLE 2

### Use of a Memantine Containing Intraocular Implant To Treat Glaucoma

A 68 year old female complains to her physician that it is becoming difficult to see. The physician determines that she has elevated intraocular pressure levels, and diagnoses her with glaucoma. An implant containing 400 μg of memantine and 600 μg of a combination of PLGA and PLA is placed in the vitreous of both of the woman's eyes using a trocar. The loss of vision is prevented for about five months after the implant procedure.

All references, articles, publications and patents and patent applications cited herein are incorporated by reference in their entireties.

While this invention has been described with respect to various specific examples and embodiments, it is to be understood that the invention is not limited thereto and that it can be variously practiced within the scope of the following claims.

1. A biodegradable intraocular implant comprising:
an anti-excitotoxic agent and a biodegradable polymer matrix that releases drug at a rate effective to sustain release of an amount of the antiexcitotoxic agent from the implant for at least about one week after the implant is placed in an eye.

2. The implant of claim 1, wherein the antiexcitotoxic agent is a glutamate receptor antagonist.

3. The implant of claim 2, wherein the antiexcitotoxic agent is an NMDA glutamate receptor antagonist.

4. The implant of claim 3, wherein the antiexcitoxic agent is an adamantine derivative, salts thereof, and mixtures thereof.

5. The implant of claim 4, wherein the glutamate receptor antagonist is memantine.

6. The implant of claim 1, further comprising an additional ophthalmically acceptable therapeutic agent.

7. The implant of claim 1, wherein the antiexcitotoxic agent is dispersed within the biodegradable polymer matrix.

8. The implant of claim 1, wherein the matrix comprises at least one polymer selected from the group consisting of polylactides, poly(lactide-co-glycolides), derivatives thereof, and mixtures thereof.

9. The implant of claim 1, wherein the matrix is substantially free of a polyvinyl alcohol.

10. The implant of claim 1, wherein the matrix comprises a poly(lactide-co-glycolide).

11. The implant of claim 1, wherein the matrix comprises a poly(D,L-lactide-co-glycolide).

12. The implant of claim 1, wherein the matrix releases drug at a rate effective to sustain release of an amount of the anti-excitotoxic agent from the implant for more than one month from the time the implant is placed in the vitreous of the eye.

13. The implant of claim 1, wherein the anti-excitotoxic agent is memantine, and the matrix releases drug at a rate effective to sustain release of a therapeutically effective amount of the memantine for a time from about two months to about six months.

14. The implant of claim 1, wherein the implant is structured to be placed in the vitreous of the eye.

15. The implant of claim 1, wherein the anti-excitotoxic agent is memantine provided in an amount from about 40% by weight to about 70% by weight of the implant, and the biodegradable polymer matrix comprises a poly(lactide-co-glycolide) in an amount from about 30% by weight to about 60% by weight of the implant.

16. The implant of claim 1 formed as a rod, a wafer, or a particle.

17. The implant of claim 1 which is formed by an extrusion process.

18. A method of making a biodegradable intraocular implant, comprising the step of: extruding a mixture of an anti-excitotoxic agent and a biodegradable polymer component to form a biodegradable material that degrades at a rate effective to sustain release of an amount of the anti-excitotoxic agent from the implant for at least about one week after the implant is placed in an eye.

19. The method of claim 18, wherein mixture consists essentially of memantine and a biodegradable polymer.

20. The method of claim 18, further comprising a step of mixing the anti-excitotoxic agent with the polymer component before the extrusion step.

21. The method of claim 18, wherein the anti-excitotoxic agent and the polymer component are in a powder form.

22. The method of claim 18, wherein the polymer component comprises a polymer selected from the group consisting of polylactides, poly(lactide-co-glycolides), and combinations thereof.

23. The method of claim 18, wherein the polymer component is substantially free of polyvinyl alcohol.

24. A method of treating an ocular condition characterized by undesirable angiogenisis in an eye of a patient, comprising the step of placing a biodegradable intraocular implant in an eye of the patient, the implant comprising an anti-excitotoxic agent and a biodegradable polymer matrix, wherein the implant degrades at a rate effective to sustain release of an amount of the anti-excitotoxic agent from the implant effective to reduce angiogenisis in the eye of the patient.

25. The method of claim 24, wherein the method is effective to treat a retinal ocular condition.

26. The method of claim 24, wherein the ocular condition includes retinal damage.

27. The method of claim 26, wherein the ocular condition is glaucoma.

28. The method of claim 26, wherein the ocular condition is proliferative vitreoretinopathy.

29. The method of claim 24, wherein the implant is placed in the posterior of the eye.

30. The method of claim 24, wherein the implant is placed in the eye with a trocar.

31. The method of claim 24, wherein the implant is placed in the eye with a syringe.

32. The method of claim 24, further comprising a step of administering a therapeutic agent in addition to the anti-excitotoxic agent to the patient.

33. The method of claim 24, wherein the anti-excitotoxic agent is memantine, salts thereof, and mixtures thereof.
